(19) United States
(12) Patent Application Publication
(10) Pub. No.: US 2026/0219338 A1
(43) Pub. Date: Jul. 30, 2026

(54) Local Coil with Hall Sensor and Magnetic Resonance Device

(71) Applicant: Siemens Healthineers AG, Forchheim (DE)

(72) Inventor: Thomas Beck, Dormitz (DE)

(73) Assignee: Siemens Healthineers AG, Forchheim (DE)

(21) Appl. No.: 19/463,222

(22) Filed: Jan. 29, 2026

(30) Foreign Application Priority Data
Jan. 30, 2025 (DE) 10 2025 103 353.6

(51) Int. Cl. G01R 33/07; G01R 33/02; G01R 33/34

(52) U.S. Cl. CPC G01R 33/07; G01R 33/0206; G01R 33/34

(57) ABSTRACT

The disclosure relates to a local coil for a magnetic resonance device, comprising a receiving element, which is configured to receive high-frequency signals in a frequency range and power range of a magnetic resonance measurement; a Hall sensor, which is mechanically connected to the local coil; and a communication unit, which has a signal connection to the Hall sensor; wherein the Hall sensor is configured to capture information about a magnetic field, and wherein the communication unit is configured to provide by means of a wireless signal connection a signal according to the information about the magnetic field. The disclosure also relates to a magnetic resonance device having a local coil according to the disclosure.

### CROSS-REFERENCE TO RELATED APPLICATIONS

The present application claims priority to and the benefit of Germany patent application no. DE 10 2025 103 353.6, filed on Jan. 30, 2025, the contents of which are incorporated herein by reference in their entirety.

### TECHNICAL FIELD

The present disclosure relates to a local coil with a Hall sensor for a magnetic resonance device.

### BACKGROUND

Magnetic resonance tomography (MRT) is a widely established imaging method in medical diagnostics. In MRT, strong magnetic fields and radiofrequency fields are used to generate image data from internal structures of an object. An essential element of a magnetic resonance tomography unit are local coils, which are placed close to the object to be examined to receive signals emitted by the internal structures.

Conventional local coils have what are known as detuning circuits, which are designed to detune receiving elements of the local coils during an excitation phase to avoid unwanted interactions with a radiofrequency field. Such detuning circuits typically comprise an active detuning circuit, which is controlled via a cable connection to the MRT system, and a passive detuning circuit, which takes effect when coils are not connected.

A problem with the use of conventional local coils is that disconnected or incorrectly positioned coils in the magnetic field of the MRT device pose a safety risk to patients under examination. Until now, it has not been easily possible to detect a defect in the active or passive detuning circuit, as only a failure of both detuning circuits can be detected by obvious technical means. This situation is not desirable for mitigating risks for patients under examination.

In addition, the occurrence of a fault in a local coil should be evident to a user or service engineer so that an appropriate response can be made. For example, this can include a repair to the local coil, excluding the local coil from a magnetic resonance measurement, or the like.

### SUMMARY

It is therefore an object of the present disclosure to provide an improved local coil that addresses and overcomes these problems. These objects are achieved according to the disclosure by the various embodiments as discussed in further detail herein, including the claims.

In a first aspect, the disclosure relates to a local coil for a magnetic resonance device, comprising: a receiving element, which is designed to receive high-frequency signals in a frequency range and power range of a magnetic resonance measurement; a Hall sensor, which is mechanically connected to the local coil; and a communication unit, which has a signal connection to the Hall sensor; wherein the Hall sensor is designed to capture information about a magnetic field, and wherein the communication unit is designed to provide by means of a wireless signal connection a signal according to the information about the magnetic field.

The receiving element is designed to receive high-frequency signals in a frequency range and power range of a magnetic resonance measurement. The receiving element can comprise an electrically conductive structure, for example a signal conductor or an antenna. The electrically conductive structure can be designed to receive electromagnetic signals in the radiofrequency range. In an embodiment, the receiving element can constitute a coupling element between electromagnetic waves guided in signal conductors and unguided electromagnetic waves, i.e. existing in free space. The receiving element can be designed to receive electromagnetic waves in any suitable range, e.g. the range of a magnetic resonance frequency of a magnetically resonant atomic nucleus. Electromagnetic waves relevant to magnetic resonance measurements can be high-frequency signals (magnetic resonance signals), which include frequencies between 1 MHz and 500 MHz, preferably between 10 MHz and 300 MHz. The magnetic resonance signals of atomic nuclei usually to be examined can have a low power of a few microwatts to several milliwatts. Such signals may be emitted by excited atomic nuclei in an object under examination during a magnetic resonance measurement.

The receiving element can take any suitable form for this purpose, for instance a coil, an antenna, a resonator, etc. The size and shape of the receiving element can be adapted to a body region to be examined to ensure optimum signal acquisition. The receiving element may for example comprise an electrically conducting wire. The wire can have an oval or polygonal cross-section. It is conceivable that the wire is designed to transfer the aforementioned powers continuously. It is also conceivable that the wire is embodied as a conductor track on a printed circuit board. The wire can be made of copper. Other electrically conducting metals are also conceivable, however, such as gold, aluminum, silver, or the like, for example.

Of course, the local coil according to the disclosure can comprise any suitable number of receiving elements. The receiving elements can be spaced apart from one another, adjacent to, or partially overlapping one another. Furthermore, the receiving elements can be arranged in the form of a grid or an array.

The receiving element can be mechanically connected or coupled to a carrier structure of the local coil. It is also conceivable that the receiving element is integrated or embedded in the carrier structure. The carrier structure can have a material that is designed to provide touch protection for a patient. In addition, the material of the carrier structure can be designed to be molded to a contour of a diagnostically relevant body region of a patient.

It is conceivable that the local coil according to the disclosure has a transmit element, which is designed to emit a high-frequency signal towards the object under examination, for instance a diagnostically relevant body region of a patient. The high-frequency signal emitted by the transmit element can lie within any suitable power range, e.g. a few Watts to several kilowatts, depending on a magnetic field strength of a magnetic field of a magnetic resonance device being used. The high-frequency signal emitted by the transmit element can be for instance a radiofrequency field or B1 magnetic field. A portion of the local coil containing the transmit element can constitute a transmit unit of the local coil, for example. The transmit element can be integrated with the receiving element or be separate from the receiving element.

A Hall sensor can constitute an electronic component that is designed to measure the magnetic field strength of a magnetic field. The Hall sensor can have crystalline doped semiconductor layers, which are connected at the sides to four electrodes. A first pair of opposite electrodes can be designed to inject a current, whereas a second pair of electrodes, which are arranged orthogonal to the first pair, can be used to pick up the Hall voltage, as is generally known. Such a Hall sensor can be designed to supply an output voltage which is proportional to a (signed) magnitude of a vector product of magnetic flux density and current when a magnetic field perpendicular to the layer passes through the Hall sensor.

In an embodiment, the Hall sensor is designed to detect the presence and/or magnetic field strength of a static magnetic field of a magnetic resonance device. It is conceivable that the information about the magnetic field comprises data about the presence and/or magnetic field strength of the magnetic field. For example, the Hall sensor can be in the form of a linear Hall sensor or a 3D Hall sensor, which can measure magnetic fields in all three spatial directions.

A communication unit can constitute an electronic component that is designed to transfer and receive data. In an embodiment, the communication unit is designed to transfer the information about the magnetic field, which information is captured by the Hall sensor, wirelessly to a further component, for example a data interface and/or a control unit of a magnetic resonance device. The communication unit can use various wireless data-transfer technologies, for instance WLAN, BLUETOOTH, and/or a variety of any suitable wireless protocols. The communication unit can also comprise functions for data encryption and error correction to ensure reliable and secure transfer. It is conceivable that the communication unit is integrated with the Hall sensor as a single component. It is also possible, however, for the communication unit to be separate from the Hall sensor and have a signal connection to the Hall sensor.

A signal connection can constitute a wired or wireless connection between two or more components that allows data and/or signals to be transferred and/or exchanged.

The signal connection between the Hall sensor and the communication unit can comprise, for example, an electrical line, an optical line, a data bus, or a wireless connection. The signal connection can be bidirectional to allow measurement data to be transferred from the Hall sensor to the communication unit and data and/or control commands to be conveyed in the opposite direction. In an embodiment, the signal connection between the Hall sensor and the communication unit is in the form of an electrical connection that is designed to transfer data captured by the Hall sensor to the communication unit. This electrical connection can be realized, for example, by conductor tracks on a printed circuit board or by cable connections.

Information about a magnetic field can comprise data that describes properties of a magnetic field. For example, the information about the magnetic field can comprise a magnetic field strength, a magnetic field direction, a spatial variation of the magnetic field, and/or changes in the magnetic field over time. It is conceivable that the information about the magnetic field can be used to determine a position of the local coil relative to a main magnetic field of a magnetic resonance device, and/or to establish whether the local coil is inside an imaging region of a magnetic resonance device.

A wireless signal connection can be designed to allow data or signals to be transferred between two or more devices without a physical connection. For example, the wireless signal connection can be designed to convey the signal provided by the communication unit to a data interface and/or a control unit of a magnetic resonance device. The wireless signal connection can use various technologies, for instance radiofrequency transmission, optical transmission, WLAN, BLUETOOTH, and the like. The choice of technology can depend on factors such as range, data rate, energy consumption, and susceptibility to noise.

In an embodiment, the communication unit comprises an electronic component which is designed to transfer data wirelessly. The communication unit can comprise, for example, a radio chip, a BLUETOOTH transmitter, or another suitable wireless communication mechanism. The communication unit can also be designed to convey the information captured by the Hall sensor about the magnetic field to an external receive unit.

The mechanical connection between the Hall sensor and the local coil can mean that the local coil is fixedly integrated in a structure, for instance in a carrier structure described above. This can advantageously ensure that the Hall sensor can measure the magnetic field strength reliably at a predetermined position of the local coil.

The described local coil can offer a number of technical advantages. The integration of the Hall sensor and the communication unit allows automatic detection of a position and/or a connection status of the local coil in an imaging region of a magnetic resonance device. For example, disconnected local coils in the magnetic field of the magnetic resonance device can be detected reliably on the basis of the signal from the communication unit and/or the information about the magnetic field, whereby the safety of patients can be increased. In addition, a workflow of a user of the local coil can be improved because the user can be informed automatically about the position and/or the connection status of the local coil. The wireless signal connection can allow flexible positioning of the local coil without the need for additional cable connections for conveying the status. This can avoid error sources associated with a signal transfer by cables. Such error sources may include, for example, a damaged or broken cable or a connector not being fully seated (e.g. plugged in).

In an embodiment, the local coil according to the disclosure comprises an energy storage unit. The energy storage unit is electrically connected to the Hall sensor and is designed to supply the Hall sensor with electrical energy.

The energy storage unit can be designed to store and provide electrical energy. The energy storage unit may e.g. be designed to supply the Hall sensor with the electrical energy needed to ensure it can function. In an embodiment, the energy storage unit is designed to store electrical energy, and to release it to the Hall sensor when required. As a result, the Hall sensor can be operated even when the local coil is not directly connected to the magnetic resonance device.

A storage capacity of the energy storage unit can be designed such that the energy storage unit can provide enough energy for a certain operating time period of the Hall sensor. For example, the operating time period can comprise several hours or even one or more days, depending on the energy consumption of the Hall sensor and the usage of the local coil.

It is conceivable that the energy storage unit comprises a charging mechanism. The charging mechanism can be designed to charge the energy storage unit. For example, this can be done through a wired connection to an external electrical power source and/or to a magnetic resonance device. It is also conceivable, however, that the charging mechanism is designed to charge the energy storage unit by means of wireless energy transfer, for example by acquiring energy from a radiofrequency field of the magnetic resonance device.

In an embodiment, the energy storage unit is designed to acquire electrical energy from a radiofrequency field by means of the receiving element. The radiofrequency field can be generated, for example, by a body coil of a magnetic resonance device. The receiving element of the local coil can act here as an antenna and convert the electromagnetic energy of the radiofrequency field into electrical energy. It is conceivable, for example, that the radiofrequency field induces in the receiving element an electric current, which can be stored by means of the energy storage unit. The energy storage unit can have a rectifier, which is designed to convert an AC voltage induced in the receiving element into a DC voltage. This DC voltage can be used to charge the energy storage unit. The energy storage unit can additionally comprise a charging regulator, which is designed to control a charging process for the energy storage unit and/or to prevent overcharging.

By providing an energy storage unit, the Hall sensor and/or the communication unit can be supplied with electrical energy without the need for a direct electrical connection to a magnetic resonance device. This can be particularly advantageous if the local coil is not connected but is in the region of influence of the radiofrequency field. Reliable sensing of the magnetic field by the Hall sensor can thereby be guaranteed even when the local coil is not connected to the magnetic resonance device. Furthermore, the energy storage unit can be designed to supply electrical power as well to other components of the local coil in addition to the Hall sensor.

The facility to acquire energy from the radiofrequency field can extend the operating time of the local coil and increase the reliability of the system. In addition, the energy storage unit can improve the flexibility in the positioning and use of the local coil, because there is no need for a constant direct power supply.

The energy storage unit can comprise a capacitor and/or a battery storage system.

A capacitor can constitute a passive electronic device. The capacitor can be designed to store electrical energy in an electric field. For example, the capacitor can be in the form of an electrolytic capacitor, a ceramic capacitor, or a super capacitor. Capacitors can be produced in various designs and sizes to achieve different capacitances and breakdown voltages. A capacitor can advantageously allow fast charging and discharging and/or serve as a compact and reliable energy source.

Alternatively or additionally, the energy storage unit can comprise a battery storage system. A battery storage system can contain one or more rechargeable or secondary batteries, which are designed to convert chemical energy into electrical energy. For example, the battery storage system can comprise a lithium-ion battery, a nickel metal hydride battery, or another suitable battery technology.

Battery storage systems can have a higher energy density than capacitors and are therefore suitable for longer operating times. In the local coil, a battery storage system can advantageously guarantee a stable energy supply over a longer time.

Depending on requirements for capacitance, charging time, and operating period, the energy storage unit can comprise a capacitor, a battery storage system, or a combination of both. A combination can exploit the advantages of both technologies, for instance the rapid charging by the capacitor and the higher storage capacity of the battery storage system.

The integration of an energy storage unit in the local coil can realize a self-sufficient energy supply for the Hall sensor and, if applicable, for further components, for instance the communication unit. As a result, the signal can be provided even when the local coil is not connected to the magnetic resonance device or to an external electrical power source.

A capacitor as part of the energy storage unit can constitute a compact and low-cost solution for short-term energy storage. A capacitor can be charged quickly when the local coil is connected to the magnetic resonance device. In addition, the energy stored in the capacitor can be available for a limited time period when the local coil is disconnected from the device.

A battery storage system as part of the energy storage unit can guarantee a supply of energy over a longer time. This can be particularly advantageous if the local coil remains disconnected from the magnetic resonance device for a prolonged period, for example during storage or transport.

The use of an energy storage unit can advantageously increase the reliability and/or functionality of the local coil by ensuring a continuous supply of energy for the Hall sensor and also for other critical components (regardless of whether the local coil is electrically connected to an external electrical power source). This can contribute to improved safety and/or efficiency when using the local coil with magnetic resonance devices.

In a further embodiment of the local coil, the energy storage unit is designed to acquire electrical energy from a radiofrequency field by means of the receiving element.

The radiofrequency field can constitute an excitation field or a B1 magnetic field, which is generated by a body coil of a magnetic resonance device. Such radiofrequency fields are typically used to excite the spins in an object to be examined, although according to the disclosure can also be used to supply energy to components of the local coil. In an embodiment, the energy storage unit is designed to store the energy acquired from the radiofrequency field, and to release it when required to other components of the local coil, for instance the Hall sensor and/or the communication unit.

By using an energy storage unit, the local coil can be operated independently of an external power supply. This allows flexible positioning of the local coil relative to the magnetic resonance device without the need for a cable connection to the power supply. In addition, the energy storage unit can ensure a continuous supply of power for the Hall sensor and/or the communication unit, even when the local coil is not connected to the magnetic resonance device or to an external electrical power source.

An energy storage unit according to the disclosure can advantageously allow energy to be acquired from the radiofrequency field, and hence wireless charging. This can extend an operating period of the local coil and/or reduce maintenance costs for the local coil.

In addition, the energy storage unit and/or the communication unit can advantageously be designed to detect from the acquisition of energy from a radiofrequency field the presence of the radiofrequency field, which can deliver additional information about the operating state of the magnetic resonance device. The communication unit can be designed to provide such additional information by means of the wireless signal connection.

The receiving element of the local coil can also be used for energy harvesting in addition to its primary function of receiving high-frequency signals for the magnetic resonance measurement. For example, the receiving element can be designed to acquire the energy contained in the radiofrequency field and convert it into electrical energy. This electrical energy can then be stored in the energy storage unit. The receiving element can be electrically connected to the energy storage unit for this purpose.

The amount of energy acquired from the radiofrequency field can depend on various factors, for instance a magnetic field strength of the radiofrequency field, an efficiency of the receiving element, and/or the energy storage unit, but also a length of exposure to the radiofrequency field. The energy storage unit and/or the receiving element can be dimensioned such that they can provide enough energy for operation of the Hall sensor and/or the communication unit over a certain time period.

An energy storage unit according to the disclosure can advantageously supply the Hall sensor and/or the communication unit with electrical energy and extend an operating time period of the Hall sensor and/or the communication unit. In addition, the energy storage unit can reduce how frequently a battery of the battery storage system is charged and/or replaced. This can increase the flexibility and/or operational capability of the Hall sensor and/or the communication unit, and/or reduce maintenance costs of the local coil.

In an embodiment of the local coil, the local coil comprises an indication unit, which has a signal connection to the Hall sensor and/or the communication unit. The indication unit is designed to provide an optical and/or acoustic signal according to the information about the magnetic field.

An indication unit can constitute a component or module designed to indicate and/or to signal information or states. For example, the indication unit can be designed to provide information about the magnetic field and/or a state of the local coil in the form of an optical and/or acoustic signal. The indication unit can be connected via a signal connection to other components of the local coil, for instance to the Hall sensor and/or the communication unit. This signal connection can make it possible for the indication unit to receive relevant information and to output a signal based thereupon. It is conceivable that the signal connection is in the form of a wired connection or a wireless connection.

The indication unit can take various forms and use different signaling methods. The indication unit can comprise, for example, light-emitting diodes (LEDs), displays, and/or other visual indicators designed to output an optical signal. Alternatively or additionally, the indication unit can comprise a loudspeaker, which is designed to produce acoustic signals such as tones, beeps, and/or spoken messages.

An optical signal can comprise a visual representation of information or states. With regard to the indication unit of the local coil, an optical signal can take any suitable and various forms. For instance, the optical signal may be a simple on/off signal of an LED, a pattern of flashes, a color change, or a more complex indicator on a display. Optical signals can use various colors to convey different states or information. For example, a green light can indicate a normal operating state, whereas a red light can point to a problem or a warning. Flashing signals can be used to draw attention to urgent situations or to distinguish between different states.

The use of optical signals in the indication unit of a local coil can be particularly advantageous in loud environments where acoustic signals might possibly not be heard. In addition, optical signals can also be discerned from a relatively large distance, allowing a user of the local coil to quickly check the status without having to be in the immediate vicinity of the local coil.

An acoustic signal can comprise an audible representation of information or states. With regard to the indication unit of the local coil, an acoustic signal can take various forms. It can be simple tones, complex tone sequences, voice messages, and/or other audible outputs.

Acoustic signals can vary in pitch, loudness, duration, and repetition rate to convey different information or states. For example, a short high-pitched tone can be used to indicate a successful operation, whereas a longer, deeper tone can point to a problem. Repeated tone sequences can be used to draw attention to urgent and/or important situations.

The use of acoustic signals in the indication unit of a local coil can be particularly advantageous in situations in which a user of the local coil cannot constantly pay attention to visual indicators. Acoustic signals can attract the attention of the user even when the user is occupied with other tasks. In addition, acoustic signals can be useful in environments in which the view of optical indicators is restricted.

The combination of optical and acoustic signals in the indication unit can allow information to be conveyed redundantly and hence more reliably. This can be particularly important in critical situations or when safety aspects are involved. The use of both signal types can also help to increase user-friendliness and reduce the likelihood of important information being overlooked or not being heard.

For example, the indication unit can comprise a light-emitting diode, which is designed to light up according to the information about the magnetic field. Alternatively or additionally, the indication unit can comprise a loudspeaker, which is designed to output a warning tone according to the information about the magnetic field. The indication unit can be designed to give direct feedback about the status of the local coil to a user of the local coil. A status of the local coil can depend on the information about the magnetic field but also on further information. For example, the status of the local coil can comprise information about a position of the local coil relative to a magnetic resonance device, e.g. a spatial position of the local coil, and also about the presence of a magnetic field and/or information about an electrical connection status of the local coil to a magnetic resonance device or to an external electrical power source.

In an embodiment, the indication unit is designed to indicate various states or information, and/or to point out various states and information. Such states or information can comprise a connection status of the local coil to a magnetic resonance device or to an external electrical power source, the magnetic field strength of a sensed magnetic field, an operating state of the local coil, but also other relevant parameters. For example, the indication unit can have a green LED light come on when the local coil is connected correctly and is operational, or activate a red LED when a problem is detected.

An indication unit according to the disclosure can offer a number of advantages. For example, the indication element can provide a user of the local coil with a quick and easy way of checking the status of the local coil without having to rely on external indicators or computer screens. This can increase the efficiency in preparing and performing magnetic resonance measurements. In addition, by directly pointing out potential problems or unwanted states of the local coil, an indication unit can help to improve the safety of both the patient and the user.

The indication unit can have a signal connection to the Hall sensor and/or the communication unit. The indication unit can be designed to provide an optical and/or acoustic signal according to the information about the magnetic field.

For example, the indication unit can comprise a loudspeaker, which can be designed to output a warning tone when the local coil is not connected to a magnetic resonance device and/or to an external electrical power source but is in a magnetic field of the magnetic resonance device. The warning tone can constitute acoustic feedback for a user of a magnetic resonance device. The user can thereby recognize quickly and easily that the local coil is not connected correctly and take appropriate action.

Alternatively or additionally, the indication unit can comprise an optical indication apparatus such as one or more light-emitting diodes (LEDs), for example. The LEDs can be designed to light up or to flash when the local coil is not connected to a magnetic resonance device and/or to an external electrical power source but is in a magnetic field of the magnetic resonance device. The optical signal can be particularly advantageous in loud environments in which acoustic signals might possibly not be heard. The indication unit can be designed to output various signal patterns or signal colors in order to indicate different states of the local coil. For example, a continuous red light can indicate a not-connected state, whereas flashing green can signal that the local coil has been connected successfully to a magnetic resonance device.

The indication unit can also be designed to indicate the strength of the detected magnetic field. This can be realized, for example, by a different pitch, loudness, or flashing frequency. A user can thereby recognize not only whether the local coil is connected properly but also whether the local coil is in a correct region of the magnetic field.

The integration of the indication unit in the local coil means that direct and immediate feedback can be made to the user without the user having to pay attention to a separate indicator on a monitor or to an indicator unit of a magnetic resonance device. This can improve a workflow associated with a magnetic resonance measurement, and/or reduce potential errors in the positioning and/or connection of the local coil.

A combination of Hall sensor, communication unit, and indication unit can provide a comprehensive solution for monitoring and indicating the state of the local coil. This can improve the safety of patients while simultaneously improving the efficiency of a workflow of a magnetic resonance measurement.

In a further embodiment of the local coil, the Hall sensor is in the form of a 3D Hall sensor.

The 3D Hall sensor can be designed to sense the magnetic flux density along three spatial directions, for instance along three mutually orthogonal spatial directions. The use of a 3D Hall sensor can allow more precise sensing of the magnetic field. The 3D Hall sensor can comprise a plurality of Hall elements, which are arranged in different orientations to measure magnetic field components of a magnetic field in the three spatial directions.

A 3D Hall sensor can constitute a semiconductor device that is designed to measure the magnetic flux density in all three spatial directions. Unlike a simple Hall sensor, which only senses components of the magnetic field that are perpendicular to the sensor surface, a 3D Hall sensor can measure x-, y- and z-components of the magnetic field simultaneously. By arranging a plurality of Hall elements in different orientations, a 3D Hall sensor can fully characterize a magnetic field in the three spatial directions.

A 3D Hall sensor can offer various advantages. For example, a more accurate characterization of a magnetic field of a magnetic resonance device can be made by measuring all three spatial components of a magnetic field. Furthermore, a 3D Hall sensor can allow a position and/or orientation of the local coil to be determined more precisely in an imaging region of the magnetic resonance device. By analyzing three-dimensional information about the magnetic field, it is possible not only to detect the presence of the local coil in the magnetic field, but also potentially to determine the spatial location and/or orientation of the local coil. Providing the Hall sensor in the form of a 3D Hall sensor can thus allow better detection and localization of the local coil in the magnetic field of the magnetic resonance device, which can contribute to greater safety and efficiency in performing magnetic resonance measurements.

The use of a 3D Hall sensor in the local coil can also provide improved sensitivity and reliability in detecting magnetic fields. For instance, even when the local coil is positioned in an unfavorable orientation with respect to the magnetic field of the magnetic resonance device, a sufficiently strong signal can advantageously be obtained at least for one measurement axis of the 3D Hall sensor.

A 3D Hall sensor can be implemented in various embodiments. One option is to integrate a plurality of individual Hall elements in one housing or carrier structure of the local coil, with each Hall element being responsible for one spatial direction. Another option is to use a single Hall element, which by means of additional structures or magnetic field concentrators allows magnetic field components to be measured along all three spatial directions.

Of course, the Hall sensor can also be embodied as a simple Hall sensor. A simple Hall sensor can be designed to measure the magnetic flux density along exactly one spatial direction. This can be advantageous for local coils for which there is no need to determine the position and orientation of the local coil precisely. For example, a simple Hall sensor can be sufficient for spatially fixed local coils (such as head coils, for example), which are typically placed in a predefined position on the patient table.

The use of a simple Hall sensor instead of a 3D Hall sensor can offer a number of advantages. For instance, a simple Hall sensor can be cheaper to produce and integrate in the local coil. In addition, a simple Hall sensor can have a lower energy consumption, which can extend not only the operating life of the energy storage unit but also an operating period of the Hall sensor. Furthermore, the signal processing and/or data transfer for a simple Hall sensor can be less complex, which can simplify the communication with the control unit.

The choice between a 3D Hall sensor and a simple Hall sensor can depend on the specific requirements for the particular local coil. For example, for local coils that allow flexible positioning on the patient, a 3D Hall sensor can be advantageous in order to allow the exact spatial position and/or orientation of the local coil to be determined. However, for local coils that have a fixed position, or those for which the exact orientation is less critical, a simple Hall sensor can be sufficient for detecting the presence of the local coil in the magnetic field.

In a further embodiment, the local coil comprises an active detuning circuit and/or a passive detuning circuit, which are electrically connected to the receiving element.

The active detuning circuit and/or the passive detuning circuit can be designed to detune the receiving element of the local coil or to alter a resonant frequency of the receiving element.

An active detuning circuit can comprise for instance an electronic circuit, which is designed to be actively controlled to alter the resonant frequency of the receiving element. The active detuning circuit can comprise, for example, a switchable capacitor and/or a switchable inductor, which can be activated or deactivated by a control signal. By activating the active detuning circuit, the resonant frequency of the receiving element can be shifted such that the receiving element is no longer tuned to an excitation frequency of a radiofrequency field.

A passive detuning circuit can comprise an electronic circuit, which responds automatically to predetermined electromagnetic fields without the need for external control. The passive detuning circuit can comprise, for example, a non-linear semiconductor, for instance a diode, in combination with a capacitor and an inductor. When a predetermined electromagnetic field arises, the passive detuning circuit can automatically shift the resonant frequency of the receiving element and thus prevent any unwanted coupling to a radiofrequency field.

The active detuning circuit and/or the passive detuning circuit can be electrically connected to the receiving element. The active detuning circuit can be designed to detune the receiving element actively when a relevant control signal is received from the control unit of the magnetic resonance device. The passive detuning circuit can be designed to detune the receiving element passively when a sufficiently strong radiofrequency field is detected.

The electrical connection of the detuning circuits to the receiving element can be realized, for example, by galvanic, capacitive, and/or inductive coupling. The active detuning circuit and/or the passive detuning circuit can be connected in parallel with the receiving element or in series with the receiving element.

The active detuning circuit can comprise a switchable semiconductor, for instance a PIN diode. Applying a forward bias to the PIN diode can reduce the resistance of the active detuning circuit and thereby detune the receiving element. The passive detuning circuit can comprise a resonant circuit containing non-linear devices such as diodes, for instance. When a certain field strength of the radiofrequency field is exceeded, the resonant circuit can start to resonate and thereby detune the receiving element.

The use of an active detuning circuit and/or a passive detuning circuit can increase the safety in a magnetic resonance measurement by avoiding unwanted interactions between the local coil and the radiofrequency field.

A combination of an active detuning circuit and a passive detuning circuit can provide redundant protection against unwanted resonances of the receiving element. This can be advantageous, for instance, if the local coil is not connected correctly to the magnetic resonance device or if the active detuning circuit fails. In these cases, the passive detuning circuit can additionally provide protection against potentially hazardous excessive increases in the field.

The integration of the detuning circuits in the local coil with Hall sensor and communication unit can implement a comprehensive safety function. The Hall sensor can detect the presence of the local coil in the magnetic field, while the detuning circuits prevent unwanted resonances. The combining of these components can advantageously achieve increased patient safety. For example, the risk of burns or other injuries caused by unwanted resonances of the local coil can be reduced. At the same time, the functionality of the local coil can be retained for the imaging because the detuning circuits are only activated when necessary.

Furthermore, information about a status of the active detuning circuit and/or the passive detuning circuit can be provided advantageously by means of an indication unit and/or the communication unit according to an above-described embodiment. A user of the local coil can thereby be informed about the status of the detuning circuits.

In a further embodiment, the local coil comprises a capacitor, which is connected in parallel with the receiving element to form a resonant circuit.

The capacitor can be electrically connected to the receiving element. Connecting the capacitor in parallel with the receiving element can form a resonant circuit. The resonant circuit can be tuned to a certain frequency. This frequency can equal the Larmor frequency of atomic nuclei to be measured. The resonant circuit can increase the sensitivity of the local coil to the high-frequency signals to be received.

The capacitor can be designed to form with the receiving element a resonant circuit. A resonant circuit can be an electrical circuit which at a certain frequency, the resonant frequency, responds particularly strongly to an arriving signal.

The forming of a resonant circuit by the parallel connection of capacitor and receiving element can have a number of advantages. For example, the resonant circuit can increase the sensitivity of the local coil to signals at the resonant frequency. Furthermore, the resonant circuit can suppress unwanted frequencies and thereby improve the signal-to-noise ratio of the local coil. The resonant frequency of the resonant circuit can be tuned precisely to the desired frequency of a magnetic resonance measurement by the choice of capacitance of the capacitor and inductance of the receiving element.

In a further embodiment of the local coil, the Hall sensor and/or the communication unit are designed to output the signal only when (e.g. triggered by) a magnetic field strength of the magnetic field exceeding a predetermined threshold value.

A magnetic field strength can be a physical quantity which states the magnetic field strength of a magnetic field at a certain point in space or averages the magnetic field strength of the magnetic field over a predetermined volume. The magnetic field strength can be measured in units of Tesla. In a magnetic resonance device, a static magnetic field can be generated, the magnetic field strength of which typically lies between 0.5 and 7 Tesla.

A predetermined threshold value can be a set value that serves as a reference or limit value. The predetermined threshold value can be characterized by a particular magnetic field strength, at and above which the Hall sensor and/or the communication unit are activated or provide a signal. It is conceivable that the predetermined threshold value for the magnetic field strength can be configured to suit the use and device specification. This allows flexible adaptation to different magnetic resonance devices having different magnetic field strengths. For example, a lower predetermined threshold value can be selected for a 1.5 Tesla system than for a 3 Tesla system.

The Hall sensor can be designed to transfer the information about the magnetic field to the communication unit only once a magnetic field strength of the magnetic field exceeds the predetermined threshold value. It is also conceivable that the communication unit is designed to provide signals only once the predetermined threshold value is exceeded. This can help to optimize the energy consumption of the local coil and to avoid unwanted signal transfers when the coil is outside the relevant magnetic field region.

Exceeding the predetermined threshold value can mean that a measured value becomes greater than the predetermined threshold value. With reference to the magnetic field strength, exceeding the predetermined threshold value can indicate that the local coil is in a region containing a sufficiently strong magnetic field.

The signal being output according to the predetermined threshold value being exceeded can advantageously reduce or prevent an unnecessary signal transfer when the local coil is outside a relevant magnetic field region, for example an imaging region of a magnetic resonance device. This can reduce the energy consumption of the local coil and minimize unwanted interference signals. Furthermore, by a signal output according to a predetermined threshold value, the local coil can automatically notify when it is in a magnetic field region relevant to operation. This can improve not only the reliability of the position detection but also the communication by the local coil. Furthermore, errors in the positioning and/or the connection of the local coil can be reduced advantageously.

Defining a predetermined threshold value for the signal transfer can be advantageous for avoiding unintentional activation of the position determination when the local coil is outside the actual magnetic resonance device. The Hall sensor and/or the communication unit can be designed to be activated or to output a signal only once a certain magnetic field strength is reached. The predetermined threshold value can here correspond to or correlate with a magnetic field strength typical for a magnetic resonance device (for example, the predetermined threshold value can also be slightly lower than a rated magnetic field strength of a magnetic resonance device).

A predetermined threshold value for the signal transfer can be combined advantageously with other described features of the local coil to facilitate comprehensive and precise monitoring of not only the position of the local coil relative to a magnetic resonance device and also the connection status of the local coil. The facility to adapt the threshold value can allow the local coil to be used effectively in different magnetic resonance devices while achieving an optimum balance between sensitivity and energy efficiency.

In a further embodiment of the local coil, the predetermined threshold value for the magnetic field strength equals any suitable minimum values, such as for instance at least 0.5 Tesla, at least 1 Tesla, at least 1.5 Tesla, at least 2.5 Tesla, at least 3 Tesla, etc. The local coil can be designed to output the signal only once the detected magnetic field strength exceeds one of these threshold values.

The predetermined threshold value can serve to guarantee reliable detection of the local coil in a magnetic field of a magnetic resonance device. The predetermined threshold value can be selected to match the typical range of the magnetic field strength of a magnetic resonance device. A threshold value of at least 0.5 Tesla can be suitable, for example, for magnetic resonance devices that have a low field strength. A threshold value of at least 1 Tesla or 1.5 Tesla can be used for magnetic resonance devices that have a moderate field strength. For high-field magnetic resonance devices, threshold values of at least 2.5 Tesla or 3 Tesla can be advantageous.

The use of a predetermined threshold value can advantageously avoid the Hall sensor and/or the communication unit being erroneously activated or providing a signal when the local coil is outside the actual imaging region. This can contribute to improved energy efficiency and reliability of the system. Furthermore, this can reduce or avoid the occurrence of false alarms and/or unintended activations.

The choice of the predetermined threshold value can depend on the sensitivity of the Hall sensor used. A more sensitive sensor can potentially work reliably already at lower field strengths, whereas a less sensitive sensor can need a higher predetermined threshold value.

Adapting the predetermined threshold value to specific properties of a magnetic resonance device and/or of the Hall sensor can achieve improved balance between reliable detection and avoiding false activations. This can contribute to improved overall functionality and reliability of the system.

Furthermore, the use of a suitable predetermined threshold value can help to improve or optimize the energy consumption of the local coil. By activating the signal transfer only once the threshold value is exceeded, unnecessary energy consumption can be avoided, which can be significant especially for battery-operated local coils or those that are self-sufficient in energy.

In a second aspect, the disclosure relates to a magnetic resonance device, which is designed to perform a magnetic resonance measurement of an object that is positioned inside an imaging region of the magnetic resonance device. The magnetic resonance device comprises a field generation unit for generating a magnetic field. The magnetic resonance device further comprises a body coil for generating a radiofrequency field. In addition, the magnetic resonance device comprises a control unit and at least one local coil according to an above-described embodiment. The communication unit of the local coil is designed to transfer the signal wirelessly to the control unit.

A wireless signal-transfer can be designed to allow data to be conveyed between the communication unit of the local coil and the control unit of the magnetic resonance device without a cable. The wireless signal-transfer can comprise various technologies, for instance WLAN, BLUETOOTH, NFC (near-field communication), or other suitable wireless protocols. The choice of transfer technology can depend on factors such as range, data rate, and energy consumption.

The wirelessly transferred signal can contain the information captured by the Hall sensor about the magnetic field, or can be based on the information about the magnetic field. The information about the magnetic field can comprise, for example, data about the magnetic field strength, the magnetic field direction, and/or other properties of the magnetic field.

The control unit can constitute a central processing unit of the magnetic resonance device. The control unit can be designed to control and/or to coordinate various functions and components of the magnetic resonance device. The control unit can comprise a microprocessor, a microcontroller, or a specialized signal processor, for example. It is also conceivable that the control unit is integrated in the magnetic resonance device or is in the form of a self-contained component.

The control unit can be equipped with a receiver or a communication interface to receive and/or process the wirelessly transferred signals from the communication unit. The control unit can be designed to process the received signals to obtain information about a state and/or a position of the local coil.

The wireless signal-transfer can offer various advantages. It can allow flexible positioning of the local coil, because there is no need for a physical connection to the control unit. In addition, the wireless signal-transfer can simplify handling of the local coil and reduce potential trip hazards posed by cables. The wireless signal-transfer can also increase reliability, because no cable connections can be damaged or come loose.

The wireless signal-transfer allows the control unit to obtain information about the status and position of the local coil without the need for a direct cable connection. This can improve the monitoring and control of the magnetic resonance device and contribute to greater safety and efficiency in performing the magnetic resonance measurements. Furthermore, the wireless signal-transfer can allow flexible positioning of the local coil inside the imaging region of the magnetic resonance device without the need for a physical connection to the control unit. This can simplify the handling and placement of the local coil. In an embodiment, the wireless signal-transfer can offer the advantage of guaranteeing safety during a magnetic resonance measurement regardless of a connection status of the local coil.

Furthermore, information from a plurality of local coils can be transferred to the control unit simultaneously by means of the wireless signal-transfer. This can be advantageous when a plurality of local coils are used during an examination.

The communication unit can be configured such that it transfers the signal only when certain conditions are met, for example when the Hall sensor detects a magnetic field strength that exceeds a predetermined threshold value. This can advantageously reduce or optimize the energy consumption of the communication unit.

The wireless communication between the communication unit and the control unit can make it easier to integrate additional functions such as, for example, conveying diagnostic information or software updates for the local coil.

The wireless signal-transfer between the communication unit and the control unit can advantageously increase the flexibility and efficiency of the magnetic resonance device by facilitating seamless communication between the local coil and the control system without the need for additional cable connections.

The magnetic resonance device according to the disclosure shares the advantages of the local coil according to the disclosure.

In an embodiment of the magnetic resonance device, the control unit is designed to obtain on the basis of the signal, information about a connection of the local coil to the magnetic resonance device.

Information about a connection of the local coil can comprise a statement as to whether the local coil is electrically and/or mechanically connected to the magnetic resonance device. For example, this information can include a binary state (connected/not connected) or a detailed status about the type and quality of the connection.

The information about the connection of the local coil (also referred to as the connection status of the local coil) can be ascertained in various ways. In one example, the control unit is designed to analyze a magnetic field strength and/or a quality of the magnetic field on the basis of the signal. If the local coil is in the magnetic field of the magnetic resonance device but is not sending an electrical signal via a cable connection, this can suggest a disconnected state of the local coil. The control unit can be designed for example to identify an electrical connection of the local coil to the magnetic resonance device on the basis of the signal and further information, for instance information about a connection of the local coil to a radiofrequency unit of the magnetic resonance device.

The control unit can comprise a processing unit or computing unit, which is designed to analyze the signal received from the communication unit of the local coil. Based on this analysis, the processing unit or computing unit can derive information about the connection status of the local coil.

The connection status of the local coil can comprise various states, for instance “connected,” “not connected,” or “partially connected.” The ascertaining of the connection status can be based on various information, for instance not only the information about the magnetic field but also a signal strength of the signal, a signal quality of the signal and/or further information.

The control unit can be designed to store, to process and, if applicable, to convey to other components of the magnetic resonance device the ascertained connection status. This information can be used for various purposes, for instance to improve or optimize the imaging, to rectify faults and/or to improve patient safety.

Furthermore, the control unit can be designed to initiate, based on the ascertained connection status, certain actions. For example, the control unit can output a warning when a local coil is detected as not connected even though it is needed for a planned examination, and/or the analysis of the signal has revealed that the local coil is in the imaging region and/or region of influence of the magnetic field of the magnetic resonance device.

Alternatively or additionally, the communication unit can be designed to convey to the control unit additional data about the connection status of the local coil to the magnetic resonance device. Such additional data can comprise information about a current flow in the local coil, a status of a detuning circuit, and/or other parameters.

A control unit that is designed to ascertain a connection status of the local coil to the magnetic resonance device can offer various advantages. For example, the control unit can help to improve patient safety by recognizing and reporting not-connected coils in the magnetic field. In addition, the control unit can improve or optimize a workflow associated with a magnetic resonance measurement by making a user of the magnetic resonance device aware in good time of connection problems with the local coil. This can prevent delays in the magnetic resonance measurement and/or potential image quality problems resulting from incorrectly connected local coils.

In addition, a magnetic resonance device according to the disclosure can increase safety for patients by ensuring that all required local coils are connected correctly or are outside an imaging region before an examination is started. This can reduce or minimize the risk of complications caused by local coils not being connected properly.

The ascertaining of the connection status by the control unit can also help to simplify the operation of the magnetic resonance device, because the user no longer has to check the connection of each individual local coil manually. This can lead to time savings and/or fewer human errors.

In an embodiment, the control unit is designed to use the information about the connection status of the local coil in order to implement adaptive measurement sequences. For example, if a certain local coil is recognized as not connected, measurement parameters can be adapted automatically to guarantee that a desired image quality is still achieved.

In a further advantageous embodiment of the magnetic resonance device, the control unit is designed to ascertain the presence of the local coil in the imaging region on the basis of the signal.

The control unit can be designed to evaluate the signal transferred wirelessly from the communication unit of the local coil in order to identify whether the local coil is inside the imaging region of the magnetic resonance device. For example, the control unit can be designed to determine from the information captured by the Hall sensor about the magnetic field whether the local coil is exposed to the magnetic field in the imaging region. This can make it possible to detect the presence of a not-connected local coil in the imaging region and to take appropriate action.

The imaging region can constitute a region or a volume of the magnetic resonance device in which can be performed magnetic resonance acquisitions from an object under examination. This region can typically be defined by the homogeneous magnetic field of a main magnet of the magnetic resonance device. The imaging region can comprise, for example, a portion of a cylindrical region, of a tunnel and/or of a patient receiving region inside the magnetic resonance device. In some embodiments, the imaging region can also correspond to a measurement region or an examination region.

The control unit can comprise a processing unit or computing unit, which is designed to analyze the signal received from the communication unit of the local coil. Based on this analysis, the control unit can determine whether the local coil is inside the imaging region of the magnetic resonance device.

The presence of the local coil in the imaging region can relate to the physical presence or positioning of the local coil inside a defined region in which the magnetic resonance measurements are performed. For example, the control unit can be designed to determine on the basis of the signal whether the local coil is inside or outside the imaging region.

Ascertaining the presence of the local coil in the imaging region of the magnetic resonance device can offer various technical advantages. One advantage is that this helps to improve patient safety by ensuring that there are no local coils left unintentionally in the imaging region. Another advantage is that automatic detection of the presence of the local coil in the imaging region can simplify a workflow for users of the magnetic resonance device and increase the efficiency of a magnetic resonance measurement.

The presence of the local coil in the imaging region can be ascertained in various ways. One option is to analyze the strength of the magnetic field detected by the Hall sensor of the local coil. If the field strength exceeds a predetermined threshold value, this can serve as an indicator that the local coil is in the imaging region.

A further option for ascertaining the presence of the local coil in the imaging region can be to analyze the spatial position and/or orientation of the local coil, provided a 3D Hall sensor is being used. For example, the control unit can be designed to evaluate position data conveyed from the communication unit and to compare it with known dimensions of the imaging region.

A magnetic resonance device according to the disclosure can advantageously allow identification of a status and/or a position of one or more local coils relative to the imaging region. This can lead to improved quality control and fewer errors in performing the magnetic resonance measurements.

In an embodiment of the magnetic resonance device, the control unit is designed to provide, on the basis of the information about the connection of the local coil to the magnetic resonance device, clearance for a magnetic resonance measurement.

The control unit can be designed to make performing a magnetic resonance measurement dependent on the local coil being connected correctly or properly to the magnetic resonance device. This can prevent a magnetic resonance measurement being carried out with a local coil that is not connected properly. Furthermore, the control unit can be designed to give clearance for a magnetic resonance measurement only when all detected local coils in the imaging region are connected properly. This can provide an additional level of safety and minimize potential risks from not-connected local coils.

A magnetic resonance measurement can comprise a series of radiofrequency pulses and gradient fields, which serve to excite and encode magnetic resonance signals from an object under examination. Giving clearance for a magnetic resonance measurement can mean starting, activating, or otherwise allowing the implementation of this series of radiofrequency pulses and gradient fields.

The information about the connection of the local coil to the magnetic resonance device can comprise a state, which states whether the local coil is electrically and/or functionally connected to the magnetic resonance device. It is conceivable that the information about the connection comprises a Boolean value (connected/not connected) or detailed status information.

The control unit can be designed to give clearance for the magnetic resonance measurement depending on the information about the connection of the local coil to the magnetic resonance device. This can mean that the control unit gives clearance for the magnetic resonance measurement only when the information about the connection of the local coil indicates that the local coil is connected properly. The control unit can also be designed to prevent or delay the measurement if the information about the connection of the local coil to the magnetic resonance device indicates that at least one local coil is not connected, or not correctly connected, to the magnetic resonance device.

By giving clearance for a magnetic resonance measurement depending on information about the connection of the local coil to the magnetic resonance device, it can be ensured that magnetic resonance measurements are performed only when the local coil is connected correctly. This can help to avoid erroneous measurements or potential safety risks, which might arise as a result of a local coil not being connected properly.

The control unit of the magnetic resonance device can comprise a logic unit or computing unit, which is designed to process and interpret the signals received from the communication unit of the local coil. This logic unit can comprise software, firmware or hardware components, which are adapted specifically for the analysis of the signals and for the decision-making about giving clearance for a magnetic resonance measurement.

The clearance given by the control unit for a magnetic resonance measurement can comprise various aspects. First, the control unit can check whether the local coil is connected properly to the magnetic resonance device. For this purpose, the control unit can be designed to perform an analysis of the information captured by the Hall sensor about the magnetic field and/or of further information, for instance information about the connection of the local coil to the control unit or to a radiofrequency unit of the magnetic resonance device, but also about a signal conveyed from the communication unit.

The control unit can be designed to activate or enable various systems and components of the magnetic resonance device depending on the information about the connection of the local coil to the magnetic resonance device. For example, this can comprise activating gradient coils, the radiofrequency unit, and/or other components required for performing a magnetic resonance measurement.

The control unit can also be designed to perform safety checks before a magnetic resonance measurement is given clearance. This can comprise checking whether the local coil is suitable for the planned measurement, whether the local coil is in a magnetic field or in a correct position relative to the magnetic resonance device (e.g. on the basis of the signal from the communication unit), and also whether further potential safety parameters are met.

The control unit can also be designed to control a user interface to indicate to a user of the magnetic resonance device information about a status of the local coil, for instance information about the presence of the local coil in a magnetic field, the information about the connection of the local coil to the magnetic resonance device, and/or the clearance for the magnetic resonance measurement. The user interface can be designed to provide visual or acoustic signals, which inform the user about the progress of the clearance process and/or the information about the status of the local coil.

Linking the clearance for a magnetic resonance measurement to the information about the connection of the local coil to the magnetic resonance device can advantageously provide an extra level of safety to reduce a risk of injury to a patient from local coils that are not being used properly. Furthermore, giving clearance as described above by means of the control unit can advantageously increase the efficiency and/or reliability of the magnetic resonance device. For example, the automatic checking and clearance can optimize a workflow associated with the magnetic resonance measurement and reduce the likelihood of operating errors. This can allow better patient safety, higher image quality, and/or more efficient use of the magnetic resonance device.

In a further embodiment, the magnetic resonance device comprises an output unit, wherein the control unit is designed to provide by means of the output unit the information about the connection of the local coil to the magnetic resonance device.

An output unit can constitute a component of the magnetic resonance device that is designed to make information visible or audible to users of the magnetic resonance device. The output unit can take various forms, for instance a screen, a display, a loudspeaker, or a combination thereof. The output unit can also be designed to give haptic feedback, for example through vibration.

The output unit can be designed to provide information about a status of the local coil, for example the information about the connection of the local coil to the magnetic resonance device. It is conceivable that the output unit is designed to provide the information about the status of the local coil in the form of a visual indicator, for instance in the form of a text message, a symbol, and/or a color code, on a screen. Alternatively or additionally, acoustic signals such as tones or spoken messages can be output.

Providing by means of the output unit the information about the connection of the local coil to the magnetic resonance device can assist the user in checking the connection status of the local coil quickly and easily. This can help to avoid errors in performing the magnetic resonance measurements and to improve the efficiency of the workflow.

In addition, the output unit can be designed to indicate further information about the local coil, for instance the position and/or orientation of the local coil in the imaging region or any malfunctions of the local coil. This can allow the user to respond quickly to potential problems and, if applicable, to take corrective action.

A combination according to the disclosure of an output unit with a control unit can advantageously increase the user-friendliness of the magnetic resonance device and help to improve patient safety by ensuring that the local coil is connected and/or positioned correctly before a magnetic resonance measurement is performed.

The output unit can comprise a visual indication apparatus such as a screen or a display. Alternatively or additionally, the output unit can comprise an acoustic output apparatus such as a loudspeaker, for example. The output unit can also comprise a haptic output apparatus such as a vibration motor, for example.

The control unit can be designed to provide the information about the connection of the local coil to the magnetic resonance device in various forms. For example, the information about the connection of the local coil to the magnetic resonance device can be output as text, a symbol, a color code and/or as an acoustic signal. The type of the output can vary depending on the connection status of the local coil.

The control unit can be designed to output by means of the output unit a green symbol and/or a confirmation tone when the local coil is connected correctly to the magnetic resonance device. When a connection of the local coil is not detected or is faulty, a red symbol and/or a warning tone can accordingly be output.

A combination according to the disclosure of control unit and output unit lets the user of the magnetic resonance device check the status of the local coil quickly and easily. This can help to avoid errors in performing the magnetic resonance measurements and to increase patient safety.

The output unit can be positioned such that the user can see it easily, for instance on an operating console or close to a patient positioning table. It is also conceivable that a plurality of output units are mounted at different points of the magnetic resonance device in order to improve the visibility of the information about the status of the local coil.

The control unit can be designed to update the information about the status of the local coil continuously or at regular intervals. This can ensure that the user is always informed about the current status of the local coil. The control unit can be designed to provide further information about the local coil by means of the output unit, for instance information such as a coil type, a serial number, and/or a time of the most recent functional check of the local coil. This further information can assist the user in ensuring that the local coil is used and maintained correctly.

The providing of the connection information via a dedicated output unit can advantageously allow clear and unambiguous feedback on the status of the local coil. This can optimize the workflows in performing magnetic resonance measurements and reduce potential sources of errors.

In a further embodiment of the magnetic resonance device, the control unit is designed to ascertain a spatial position of the local coil on the basis of the signal.

The local coil can comprise a 3D Hall sensor according to an above-described embodiment. The control unit can be designed to evaluate the information about the magnetic field, which is provided by the 3D Hall sensor and provided as a signal by means of the communication unit, in order to ascertain a spatial position and/or orientation of the local coil. The signal can contain information about the magnetic field strength and/or direction of the magnetic field at the position of the local coil. It is conceivable that the control unit is designed to ascertain from this information the exact spatial position and/or orientation of the local coil in three-dimensional space.

A spatial position can comprise a three-dimensional location and/or orientation of the local coil in space. For example, the ascertaining of the spatial position can comprise determining x-, y- and z-coordinates and rotational angles about respective axes.

The ascertaining of the spatial position of the local coil can be advantageous in order to determine an exact location of the local coil relative to a patient and/or to the magnetic resonance device. This can be useful for various purposes, for instance optimizing the image quality, adapting sequences of the magnetic resonance measurement, and/or improving patient safety.

The control unit or a computing unit of the control unit can be designed to use various algorithms or calculation methods in order to ascertain on the basis of the information about the magnetic field or on the basis of the signal, the spatial position of the local coil. Such an algorithm or calculation method can comprise, for example, triangulation techniques, Kalman filters, and/or other mathematical models.

By ascertaining the spatial position of the local coil, the magnetic resonance device can advantageously achieve better supervision of the positioning of the local coil. This can lead to more precise imaging and/or better diagnostic accuracy.

Determining according to the disclosure the spatial position of the local coil can offer various advantages. For example, by means of a 3D Hall sensor, the control unit can not only identify the presence of the local coil in the imaging region of the magnetic resonance device but also determine the exact position and/or orientation of the local coil in a three-dimensional space.

The control unit can be designed to process the information captured by the 3D Hall sensor about the magnetic field in order to calculate the x-, y- and z-coordinates of the local coil relative to the magnetic resonance device. Such position information can be used for various purposes. For example, the knowledge of the exact position of the local coil can be used to optimize imaging parameters of a magnetic resonance measurement. The control unit can be designed to adapt the gradient fields and/or radiofrequency fields on the basis of the position of the local coil in order to improve the image quality in the region covered by the local coil.

Furthermore, the position information for the local coil can be used for automatic registration and calibration of the magnetic resonance device.

It is also conceivable that the control unit is designed to determine positions of different local coils relative to each other and also relative to a main magnet of the magnetic resonance device, thereby allowing a precise reconstruction of the image data.

Ascertaining the spatial position of the local coil can help to improve patient safety. For example, the control unit can be designed to check whether the local coil is in an intended anatomical position and/or is connected properly. The control unit can advantageously output a warning if errors and/or deviations from a protocol or usual procedures are identified.

In addition, the position information can be used advantageously for advanced imaging techniques. For example, in the case of parallel imaging, sensitivity profiles of individual coil elements can be calculated on the basis of their exact position, leasing to improved image reconstruction.

Ascertaining the spatial position of the local coil can advantageously allow improved automation of an imaging process, optimization of the image quality, and/or increased patient safety in the magnetic resonance device.

### BRIEF DESCRIPTION OF THE DRAWINGS

Exemplary embodiments of the disclosure are illustrated in the drawings and described in more detail below. The same reference signs are used for the same features in different figures, in which:

FIG. 1 illustrates an example representation of an embodiment of a magnetic resonance device according to the disclosure;

FIG. 2 illustrates an example representation of an embodiment of a local coil according to the disclosure;

FIG. 3 illustrates an example representation of an embodiment of a local coil according to the disclosure having a Hall sensor and a communication unit;

FIG. 4 illustrates an example representation of an embodiment of a local coil according to the disclosure having an energy storage unit;

FIG. 5 illustrates an example representation of an embodiment of a local coil according to the disclosure having a control unit; and

FIG. 6 illustrates an example representation of an embodiment of a local coil according to the disclosure having an indication unit.

### DETAILED DESCRIPTION OF THE DISCLOSURE

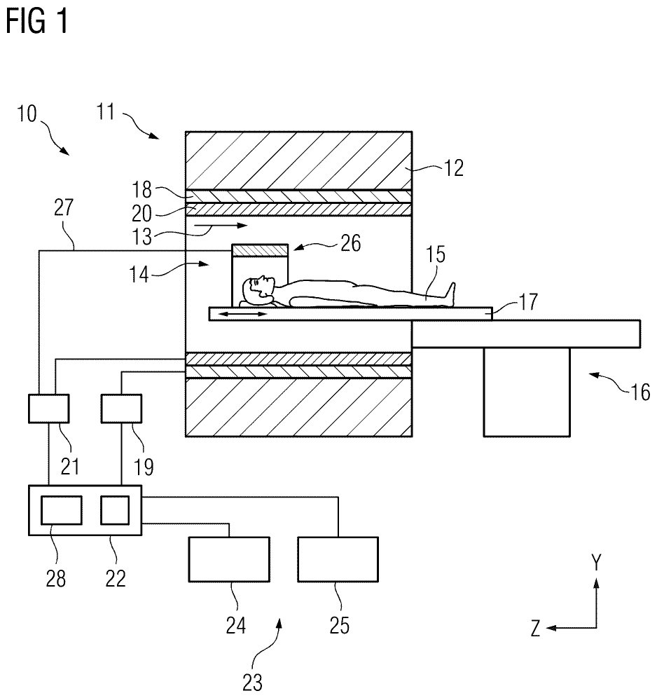

FIG. 1 illustrates an example representation of an embodiment of a magnetic resonance device according to the disclosure. For instance, FIG. 1 shows schematically a possible embodiment of a magnetic resonance apparatus 10 according to the disclosure having a local coil 26 according to the disclosure. The magnetic resonance apparatus 10 comprises a magnet unit 11, which has, for example, a permanent magnet, an electromagnet, or a superconducting main magnet 12 for generating a strong and homogeneous main magnetic field 13 (B0 magnetic field). The magnetic resonance apparatus 10 also comprises a patient receiving region 14 for accommodating a patient 15. In the present exemplary embodiment, the patient receiving region 14 is shaped as a cylinder and is enclosed in a circumferential direction by the magnet unit 11. In principle, however, it is also conceivable that the magnetic resonance apparatus 10 and/or the patient receiving region 14 differ in design from this example.

The magnetic resonance apparatus 10 may e.g. have a patient positioning apparatus 16, which is designed to position the patient 15 in the patient receiving region 14. The patient positioning apparatus 16 can comprise for this purpose a patient table 17, which is designed to be able to move inside the patient receiving region 14.

In the embodiment shown in FIG. 1, the magnet unit 11 has a gradient coil 18 for generating gradient magnetic fields, which are used for spatial encoding during a magnetic resonance measurement. The gradient coil 18 is controlled by a gradient control unit 19 of the magnetic resonance apparatus 10. The magnet unit 11 can also comprise a radiofrequency antenna, which in the example shown is in the form of a body coil 20 that is fixedly integrated in the magnetic resonance apparatus 10. The body coil 20 is designed to excite atomic nuclei located in the main magnetic field 13 generated by the main magnet 12. The body coil 20 is controlled by a radiofrequency unit 21 of the magnetic resonance apparatus 10, and radiates high-frequency signals (also known as a radiofrequency field or B1 magnetic field) into an examination space, which is largely formed by a patient receiving region 14 of the magnetic resonance apparatus 10. The body coil 20 can also be designed to receive magnetic resonance signals.

In the present case, the magnetic resonance apparatus 10 has a control unit 22 (also referred to herein as a controller, control circuitry, or a control computer), which is designed to control and/or coordinate the main magnet 12, the gradient control unit 19, and the radiofrequency unit 21. The control unit 22 may e.g. be designed to control an implementation of a sequence, for instance an imaging gradient echo sequence, a TSE sequence, or a UTE sequence. The control unit 22 can comprise a computing unit 28 (also referred to herein as processing circuitry), which is configured to analyze digitized magnetic resonance signals captured during a magnetic resonance measurement.

In addition, the magnetic resonance apparatus 10 can comprise a user interface 23, which has a signal connection to the control unit 22. Control information such as imaging parameters and reconstructed magnetic resonance images, for example, can be displayed to a user on an output unit 24 (also referred to herein as output circuitry or simply an output), for instance on at least one monitor of the user interface 23. In addition, the user interface 23 has an input unit 25 (also referred to herein as input circuitry or simply an input), by means of which the user can enter parameters for magnetic resonance imaging.

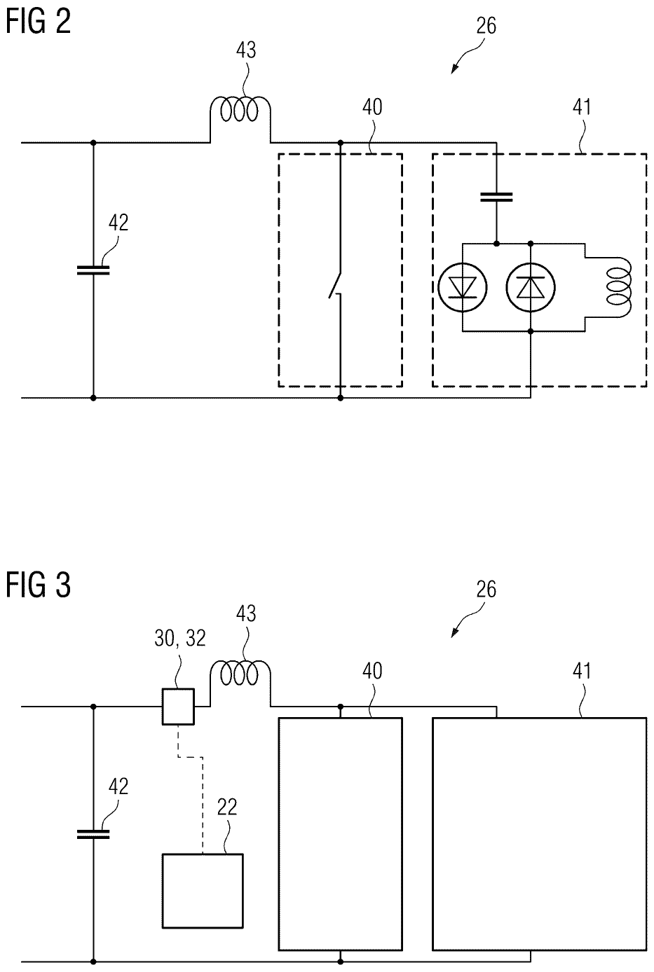

The magnetic resonance apparatus 10 comprises a local coil 26 according to the disclosure, which in the present example is positioned at a head of the patient 15. The local coil 26 is designed to capture magnetic resonance signals from a volume of the head region and to transfer them to the magnetic resonance apparatus 10. The local coil 26 preferably has an electrical connecting line 27, which provides a signal connection between a receiving element 43 (also referred to herein as a receiver, an antenna, or receiving circuitry; see FIG. 2 to 6) of the local coil and a receive unit of the magnetic resonance apparatus 10, e.g. the radiofrequency unit 21 and/or the control unit 22. A communication unit 32 (also referred to herein as communication circuitry or a communication interface) of the local coil 27 (see FIG. 3 to 6) can be connected to the magnetic resonance apparatus 10 by means of a wireless signal connection.

The control unit 22 and/or the computing unit 28 can be designed to obtain, on the basis of a signal from the communication unit 32 (see FIG. 3 to 6), information about a connection of the local coil 26 to the magnetic resonance apparatus 10 (also referred to herein as connection data). It is also conceivable that the control unit 22 and/or the computing unit 28 are designed to ascertain the presence of the local coil 26 in the imaging region 14 on the basis of the signal.

In an embodiment, the control unit 22 and/or the computing unit 28 are designed to provide, on the basis of the information about the connection of the local coil 26 to the magnetic resonance device 10, clearance for a magnetic resonance measurement, and/or to provide by means of the output unit the information about the connection of the local coil 26 to the magnetic resonance device 10. It is also conceivable that the local coil comprises a 3D Hall sensor, and the control unit 22, and/or the computing unit 28 are designed to ascertain a spatial position of the local coil 26 on the basis of a signal resulting from the 3D Hall sensor.

In a similar way to the body coil 20, the local coil 26 can also be designed to excite atomic nuclei and to receive magnetic resonance signals. For example, a transmit unit of the local coil 26 is driven by the radiofrequency unit 21 to emit high-frequency signals.

The magnetic resonance device 10 shown can obviously comprise further components that are usually present in magnetic resonance devices. It is conceivable that the magnetic resonance device 10 has, instead of the cylindrical construction, a C-shaped, triangular, or asymmetrical construction of the magnetic-field generating components. In an embodiment, the magnetic resonance device 10 can be in the form of a dedicated scanner, which is designed to perform magnetic resonance imaging of an extremity or a head region of a standing or sitting patient 15.

FIG. 2 illustrates an example representation of an embodiment of a local coil according to the disclosure. For instance, FIG. 2 shows a schematic representation of an embodiment of a local coil 26 for a magnetic resonance device 10. The local coil 26 can comprise a receiving element 43, which is designed to receive high-frequency signals in a frequency range and power range of a magnetic resonance measurement. A capacitor 42 can be connected in parallel with the receiving element 43 to form a resonant circuit.

The local coil 26 can comprise an active detuning circuit 40 and/or a passive detuning circuit 41. The active detuning circuit 40 and the passive detuning circuit 41 can be electrically connected to the receiving element 43. The passive detuning circuit 41 can comprise a capacitor, two diodes connected in anti-parallel, and an inductor.

The active detuning circuit 40 and the passive detuning circuit 41 can be connected in parallel with each other and in series with the receiving element 43. This arrangement can allow detuning of a resonant circuit of the local coil 26 under different conditions. The active detuning circuit 40 can be controlled externally (for example via the electrical connecting line 27), whereas the passive detuning circuit 41 can work passively on the basis of currents induced in the circuit.

The circuit shown in FIG. 2 of the local coil 26 can be designed to allow magnetic resonance signals to be received when it is tuned (or resonant), and to prevent unwanted interactions with the transmit field when it is detuned. The combination of active detuning circuit 40 and passive detuning circuit 41 can provide redundancy and improve the safety and reliability of the local coil 26 in a magnetic resonance imaging environment.

FIG. 3 illustrates an example representation of an embodiment of a local coil according to the disclosure having a Hall sensor and a communication unit. For instance, FIG. 3 shows a schematic representation of a further embodiment of a local coil 26 according to the disclosure for a magnetic resonance device 10. In this case, the local coil 26 comprises a receiving element 43, which is designed to receive high-frequency signals in a frequency range and power range of a magnetic resonance measurement. In addition, the local coil comprises a capacitor 42, which is connected in parallel with the receiving element 43 and allows a resonant circuit to be formed.

The local coil 26 further comprises a Hall sensor 30 and a communication unit 32, which in the present embodiment are combined in one unit. The Hall sensor 30 can be designed to capture information about a magnetic field. The communication unit 32 can have a signal connection to the Hall sensor 30 and be designed to provide, by means of a wireless signal connection (dashed line), a signal encoding the information about the magnetic field (also referred to herein as magnetic field data). In the example shown in FIG. 3, the communication unit 32 is designed to transfer (e.g. transmit) the signal to the control unit 22 of the magnetic resonance device 10. The communication unit 32 can use Wi-Fi-based communication, for example, to convey the signal.

The local coil 26 can further comprise an active detuning circuit 40 and/or a passive detuning circuit 41, which can be electrically connected to the receiving element 43. These detuning circuits 40, 41 can be used to detune the local coil 26 when required.

The control unit 22 can be designed to receive information from the Hall sensor 30 and from the communication unit 32 to facilitate supervision and/or monitoring of the local coil 26.

It is conceivable that the Hall sensor 30 and/or the communication unit 32 are integrated in the electrical circuit of the local coil 26. It is also conceivable, however, that the Hall sensor 30 and/or the communication unit 32 are separate from, or electrically isolated from, the electrical circuit, and are connected or coupled to the receiving element 43 by a housing and/or a carrier structure of the local coil 26 (not shown).

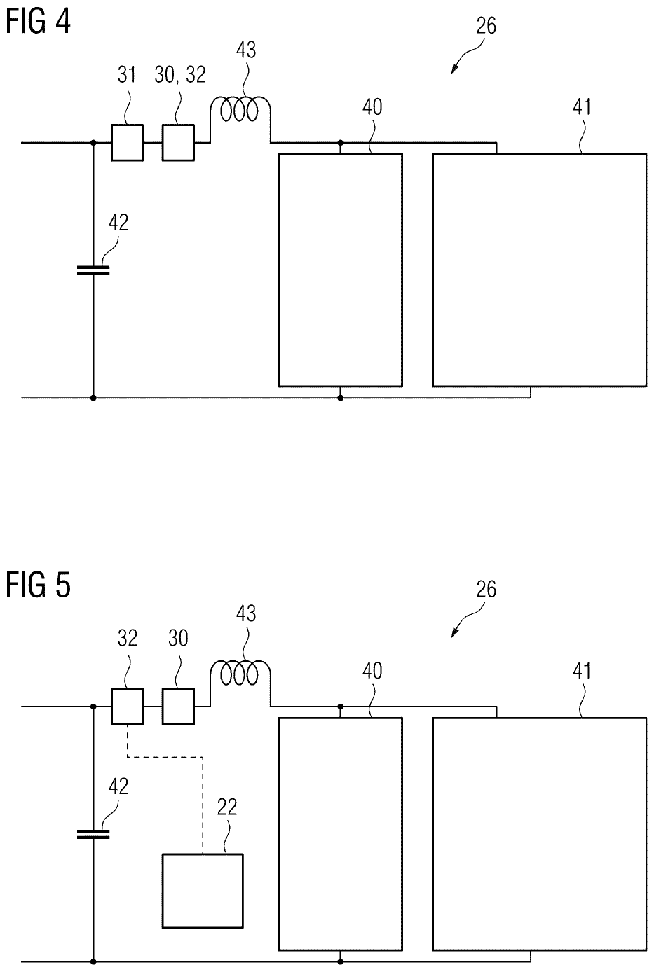

FIG. 4 illustrates an example representation of an embodiment of a local coil according to the disclosure having an energy storage unit. For instance, FIG. 4 shows a schematic representation of a further embodiment of a local coil 26 according to the disclosure for a magnetic resonance device 10. The local coil 26 comprises a receiving element 43 and a capacitor 42, which can have a similar design to the examples of FIG. 2 or 3. It is also conceivable that the local coil 26 has an active detuning circuit 40 and/or a passive detuning circuit 41 according to an above-described example.

In addition, the local coil 26 comprises a Hall sensor 30, which is mechanically connected to the local coil 26. The Hall sensor 30 is designed to capture information about a magnetic field (also referred to herein as magnetic field data). The communication unit 32 can have a signal connection to the Hall sensor 30 and be designed to provide, by means of a wireless signal connection, a signal encoding the information about the magnetic field.

In the embodiment shown in FIG. 4, the local coil 26 additionally comprises an energy storage unit 31, which is electrically connected to the Hall sensor 30. The energy storage unit 31 can be designed to supply the Hall sensor 30 with electrical energy. The energy storage unit 31 can comprise, for example, a capacitor and/or a battery storage system.

The energy storage unit may for instance be electrically connected to the receiving element 43. For example, the energy storage unit can be designed to acquire energy from a radiofrequency field by means of the receiving element 43 and/or a further receiving element (not shown), and to store this energy. It is also conceivable, however, that the energy storage unit 31, and also the Hall sensor 30 and the communication unit 32, form a circuit that is electrically isolated from the receiving element 43. It is conceivable that the energy storage unit 31, and also the Hall sensor 30 and the communication unit 32, are electrically connected to the radiofrequency unit 21, the control unit 22, or an external electrical power source by means of the electrical connecting line 27 or a line that is electrically isolated from the electrical connecting line 27.

The arrangement of components shown in FIG. 4 can allow the local coil 26 to act both as a receiver for magnetic resonance signals and as a self-monitoring device, which can detect when it is present in a magnetic field. The local coil 26 can provide this information wirelessly by means of the communication unit 32, thereby allowing an improvement in safety and a workflow associated with a magnetic resonance measurement.

FIG. 5 illustrates an example representation of an embodiment of a local coil according to the disclosure having a control unit. For example, FIG. 5 shows a schematic representation of a further embodiment of a local coil 26 according to the disclosure for a magnetic resonance device 10. The local coil 26 can comprise a receiving element 43 and a capacitor 42 according to an above-described embodiment.

The local coil 26 also comprises a Hall sensor 30, which is designed to capture information about a magnetic field (e.g. the magnetic field data). The Hall sensor 30 can be connected mechanically to the local coil 26 in any suitable way. In the example shown, the local coil 26 additionally has a communication unit 32, which is designed to be self-contained and is connected to the Hall sensor 30 by means of a signal connection. The communication unit 32 is designed to provide by means of a wireless signal connection a signal according to the information about the magnetic field.

The local coil 26 can comprise an active detuning circuit 40 and/or a passive detuning circuit 41 according to an above-described embodiment.

A control unit 22 can be connected to the communication unit 32 by means of a wireless signal connection (dashed line). The control unit 22 can be designed to receive and process the signal provided by the communication unit 32.

The arrangement of components shown in FIG. 5 can allow the local coil 26 both to act as a receiver for magnetic resonance signals and to detect and communicate the presence of the local coil 26 in a magnetic field. The Hall sensor 30 can detect the presence and position of the local coil 26 inside the magnetic resonance device, while the communication unit 32 can transfer this information wirelessly to the control unit 22.

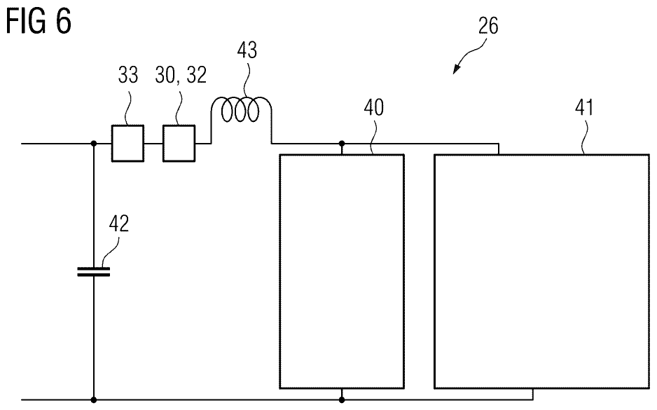

FIG. 6 illustrates an example representation of an embodiment of a local coil according to the disclosure having an indication unit. For example, FIG. 6 shows a schematic representation of a further embodiment of a local coil 26 for a magnetic resonance device 10. The local coil 26 can comprise a receiving element 43 and a capacitor 42 according to an above-described embodiment.

The local coil 26 comprises a Hall sensor 30, which can be connected mechanically to the local coil 26 in any way. The Hall sensor 30 is designed to capture information about a magnetic field. The local coil 26 further comprises a communication unit 32, which has a signal connection to the Hall sensor 30. The communication unit 32 can be designed to provide a signal according to the information about the magnetic field by means of a wireless signal connection according to an above-described embodiment.

In addition, the local coil 26 comprises an indication unit 33, which has a signal connection to the Hall sensor 30 and/or to the communication unit 32. The indication unit 33 can be designed to provide an optical and/or acoustic signal encoding the information about the magnetic field.

In an embodiment, the indication unit 33 comprises am LED and/or a loudspeaker, which are designed to output a signal encoding the information about the magnetic field. For example, the indication unit 33 can be designed to output an acoustic and/or optical signal as soon as the Hall sensor 30 detects a magnetic field. It is also conceivable that the indication unit 33 is designed to output an acoustic and/or optical signal as soon as the Hall sensor 30 detects a magnetic field that has a magnetic field strength above a predetermined threshold value. Furthermore, the indication unit 33 can be designed to output the acoustic and/or optical signal only when the Hall sensor 30 detects a magnetic field and the local coil 26 is not connected, or not connected properly, by means of the electrical connecting line 27 to the magnetic resonance device 10 (see FIG. 1).

The local coil 26 can comprise an active detuning circuit 40 and/or a passive detuning circuit 41 according to an above-described embodiment.

The arrangement of components shown in FIG. 6 can allow the local coil 26 to act both as a receiver for magnetic resonance signals and as a self-monitoring device, which can detect when it is present in a magnetic field. The local coil 26 can transfer this information wirelessly via the communication unit 32, which can potentially improve safety and the workflow in magnetic resonance imaging procedures.

The schematic representations shown in the described figures are not shown to scale in any way and do not depict relative sizes.

It is pointed out that the apparatuses described in detail above involve merely exemplary embodiments, which can be modified by a person skilled in the art in many different ways without departing from the scope of the disclosure. For example, features of individual embodiments can be combined with features of other embodiments unless such a combination is explicitly ruled out.

In addition, the use of the indefinite article “a” or “an” does not rule out the possibility of there also being more than one of the features concerned. Likewise, the terms “unit” and “element” do not exclude the possibility that the components in question consist of a plurality of interacting sub-components, which may also be spatially distributed if applicable.

Independent of the grammatical term usage, individuals with male, female or other gender identities are included within the term.

Additionally, the various components described herein may be referred to as “units.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently or configured to execute instructions or computer programs that are stored on a suitable computer-readable medium. Regardless of the particular implementation, such units, etc., as applicable and relevant, may alternatively be referred to herein as “circuitry,” “controllers,” “processors,” or “processing circuitry,” or alternatively as noted herein.

What is claimed is:

1. A local coil for a magnetic resonance device, comprising:
a receiver configured to receive electromagnetic signals during a magnetic resonance measurement;
a Hall sensor mechanically coupled to the local coil; and
communication circuitry communicatively coupled to the Hall sensor,
wherein the Hall sensor is configured to measure magnetic field data associated with a magnetic field during the magnetic resonance measurement, and
wherein the communication circuitry is configured to wirelessly transmit a signal encoding the magnetic field data.

2. The local coil as claimed in claim 1, further comprising:
an energy storage unit electrically coupled to the Hall sensor,
wherein the energy storage unit is configured to supply the Hall sensor with electrical energy.

3. The local coil as claimed in claim 2, wherein the energy storage unit comprises a capacitor and/or a battery storage system.

4. The local coil as claimed in claim 2, wherein the energy storage unit is configured to acquire, via the receiver, electrical energy from a radiofrequency field of the magnetic resonance device.

5. The local coil as claimed in claim 1, further comprising:
output circuitry communicatively coupled to the Hall sensor and/or to the communication circuitry,
wherein the output circuitry is configured to provide an optical and/or acoustic signal encoding the magnetic field data.

6. The local coil as claimed in claim 1, wherein the Hall sensor comprises a 3D Hall sensor.

7. The local coil as claimed in claim 1, further comprising:
a detuning circuit electrically coupled to the receiver.

8. The local coil as claimed in claim 1, wherein the communication circuitry is configured to transmit the signal based on a magnetic field strength of the magnetic field exceeding a predetermined threshold value.

9. The local coil as claimed in claim 8, wherein the predetermined threshold value is at least 0.5 Tesla.

10. A magnetic resonance device configured to perform a magnetic resonance measurement of an object positioned inside an imaging region of the magnetic resonance device, the magnetic resonance device comprising:
a field generator configured to generate a magnetic field;
a body coil configured to generate a radiofrequency (RF) field;
a controller; and
a local coil, comprising:
a receiver configured to receive electromagnetic signals during the magnetic resonance measurement;
a Hall sensor mechanically coupled to the local coil; and
communication circuitry communicatively coupled to the Hall sensor,
wherein the Hall sensor is configured to measure magnetic field data associated with the generated magnetic field, and
wherein the communication circuitry is configured to wirelessly transmit a signal encoding the magnetic field data to the controller.

11. The magnetic resonance device as claimed in claim 10, wherein the controller is configured to obtain, based upon the transmitted signal, connection data with respect to a connection between the local coil and the magnetic resonance device.

12. The magnetic resonance device as claimed in claim 10, wherein the controller is configured to ascertain a presence of the local coil in the imaging region based upon the transmitted signal.

13. The magnetic resonance device as claimed in claim 11, wherein the controller is configured to provide, based on the connection data, a clearance to perform the magnetic resonance measurement.

14. The magnetic resonance device as claimed in claim 11, further comprising
output circuitry,
wherein the controller is configured to provide, via the output circuitry, the connection data.

15. The magnetic resonance device as claimed in claim 10, wherein the Hall sensor comprises a 3D Hall sensor, and
wherein the controller is configured to ascertain a spatial position of the local coil based upon the transmitted signal.
